(19) United States
(12) Patent Application Publication
(10) Pub. No.: US 2025/0154228 A1
(43) Pub. Date: May 15, 2025

(54) HIGH-TRANSDERMAL-ABSORPTION PEPTIDE AND RECOMBINANT COLLAGEN CONSTRUCTED BY MEANS OF REPETITIONS OF PEPTIDE

(71) Applicant: XI'AN GIANT BIOGENE TECHNOLOGY CO., LTD, Shaanxi (CN)

(72) Inventors: Daidi FAN, Shaanxi (CN); Weigang YUWEN, Shaanxi (CN); Zhiguang DUAN, Shaanxi (CN); Ru XU, Shaanxi (CN); Jianya YAN, Shaanxi (CN); Liping JIA, Shaanxi (CN); Chanyuan YANG, Shaanxi (CN)

(73) Assignee: XI'AN GIANT BIOGENE TECHNOLOGY CO., LTD, Shaanxi (CN)

(21) Appl. No.: 18/833,622

(22) PCT Filed: Jan. 19, 2023
(86) PCT No.: PCT/CN2023/073220

§ 371 Date: Jul. 26, 2024

(30) Foreign Application Priority Data
Jan. 27, 2022 (CN) 202210100932.8

(51) Int. Cl. C07K 14/78

(52) U.S. Cl. CPC C07K 14/78

(57) ABSTRACT

The present invention relates to a high-transdermal-absorption peptide and I-type recombinant collagen constructed by means of repetitions of the peptide. The high-transdermal-absorption I-type recombinant collagen of the present invention is formed by means of multiple repetitions of the twenty-first peptide amino acid sequence from natural human I-type collagen as a repeating unit, wherein the sequence of the twenty-first peptide amino acid is as shown in SEQ ID No.: 1, and the number of repetitions is at least three. The high-transdermal-absorption I-type recombinant collagen can be used as a raw material for producing various collagen products, such as injections, facial fillers, dressings, cosmetics, healthcare foods, tissue engineering materials, collagen sponges, etc.

### TECHNICAL FIELD

The application belongs to the field of biotechnology, and in particular relates to a high-transdermal-absorption peptide and recombinant collagen constructed by means of repetitions of peptide, and use thereof.

### BACKGROUND ART

Collagen protein is a biological polymer protein, the main component of animal connective tissue, and the most abundant and widely distributed functional protein in mammals, accounting for 25% to 30% of the total protein. Collagen protein is closely related to the formation and maturation of tissues, the transmission of information between cells, joint lubrication, wound healing, calcification, blood coagulation and aging, etc., is one of the most critical raw materials in the biotechnology industry, and is widely used in medical materials, cosmetics and food industries.

Collagen protein, also known as collagen, is an important protein component of connective tissue that supports and protects the body, and is also the most abundant structural protein in many vertebrates and invertebrates, which provides mechanical strength to bones, tendons, cartilage and skin. Collagen is one of the most abundant proteins in mammals, accounting for about 20%-30% of the total protein in the body. It mainly exists in skin, bones, muscles, tendons, soft tissues, etc., wherein, about 70%-80% of the extracellular matrix of the skin is collagen. Collagen is closely related to the formation and maturation of tissues, the transmission of information between cells, joint lubrication, wound healing, calcification, blood coagulation and aging, etc., is one of the most critical raw materials in the biotechnology industry, and is widely used in the field of medical treatment or beauty.

When collagen is used in medical dressings, cosmetics, etc., it is hoped that collagen has good transdermal absorption performance from the perspective of better biological activity. However, collagen is a macromolecular bioactive substance, which itself is not easily absorbed through the skin. Therefore, in previous studies, how to promote the transdermal absorption of collagen has become a research hotspot. For example, in order to promote the transdermal absorption of collagen molecules with a large molecular weight, a mask towel-type collagen dressing may be used, that is, the collagen solution is impregnated in a solid carrier such as non-woven fabric, then sealed in a container, taken out before use, and applied on the face. In the above-mentioned mask towel-type collagen dressing, the collagen solution is impregnated in the solid carrier with liquid absorption property, which can make more collagen solution contained and delay the collagen solution drying up, and thus the interaction time between the collagen solution and the skin surface can be prolonged and promotes the penetration of collagen molecules with large molecular weight.

In recent years, with the wide application of genetic engineering technology, researchers have created various types of recombinant collagen. For example, recombinant collagen may be constructed by selecting and repeating a short amino acid sequence derived from natural human collagen. The recombinant collagen constructed in this way has advantages such as low immunogenicity, high biological activity, and good stability. Theoretically, the transdermal absorption performance of this recombinant collagen may be related to its amino acid sequence, which has not been clearly confirmed. Especially for how to design the short amino acid sequence as a repeating unit to make the constructed recombinant collagen has better transdermal absorption performance, there is no theory in the prior art that can be used as a guide.

### SUMMARY

In order to solve the above-mentioned technical problems in the prior art, the inventors conducted in-depth research, and as a result obtained a short amino acid sequence derived from natural human type I collagen and a type I recombinant collagen constructed using the short amino acid sequence as a repeating unit, the type I recombinant collagen having excellent transdermal absorption performance, thereby completing the present application.

That is, the present application comprises:

1. A polypeptide derived from natural human type I collagen, having an amino acid sequence as shown in SEQ ID No.: 1 (GAPGAPGSQGAPGLQGMPGER).

2. A type I recombinant collagen, forming by multiple repetitions of a short amino acid sequence derived from natural human type I collagen as a repeating unit, wherein, the short amino acid sequence is shown in SEQ ID No.: 1 (GAPGAPGSQGAPGLQGMPGER), and times of the repetitions is three or more.

3. The type I recombinant collagen according to item 2, wherein, the times of the repetitions is 5-150, preferably 10-100.

4. The type I recombinant collagen according to item 2, further having a tag facilitating purification thereof, the tag being a His tag, a Flag tag or a c-Myc tag.

5. Use of the type I recombinant collagen according to any one of items 2-4 in the preparation of a collagen product.

6. The use according to item 5, wherein the collagen product is selected from the group consisting of an injection, a facial filler, a dressing, a cosmetic, a health food, a tissue engineering material and a collagen sponge.

### BRIEF DESCRIPTION OF THE DRAWINGS

FIG. 1 is the SDS-PAGE protein electrophoresis diagram of purified recombinant collagens P-1˜P-4.

FIG. 2 is the SDS-PAGE protein electrophoresis diagram of purified recombinant collagens D-1˜D-4.

### DETAILED DESCRIPTION

The present application will be described in detail through specific examples below. It should be pointed out that these descriptions are only illustrative and do not limit the scope of the present application.

We firstly screened various short amino acid sequences derived from natural human type I collagen (such short amino acid sequences are 100% homologous to natural human type I collagen, which can avoid problems such as immunogenicity of foreign substances), to obtain a short amino acid sequence with excellent transdermal absorption performance. Then, type I recombinant collagen with various molecular weights was constructed using the short amino acid sequence as a repeating unit, and its transdermal absorption performance was verified. Therefore, the type I recombinant collagen with excellent transdermal absorption performance was obtained.

It should also be noted that the collagen having good adhesion-promoting effect is an important reason why it may be widely used in implanted medical devices. The implanted collagen medical devices can promote the fibroblasts, adipocytes, dermal cells, etc. to migrate to the implanted device, and exhibit cell adhesion, promoting cell growth and other characteristics, achieving rapid repair. GER tripeptide is a known tripeptide with adhesion effect, and collagen containing this tripeptide exhibits better adhesion-promoting effect. In order to ensure the physiological activity of the obtained type I recombinant collagen with excellent transdermal absorption performance, when screening the short amino acid sequence with high transdermal absorption performance, the natural short amino acid sequence containing GER tripeptide was preferentially selected, and then was repeated to construct a new type I recombinant collagen.

### Example 1: Obtaining Polypeptide with High Transdermal Absorbability

### 1) Preparation of Collagen Peptide

The telopeptide amino acid sequences at both ends of the natural amino acid sequence of type I collagen were removed, remaining 1057 amino acids (162-1219) in the middle. The fragments containing GER tripeptide were screened by amino acid analysis software, using 21 amino acids as the basic unit. Considering that the amino acid composition with GER tripeptide as the middle part may have influence on efficacy, only the amino acid sequences with GER as the beginning and end of the peptide segment were screened. A total of 18 polypeptide fragments were screened out. The 18 short peptides were synthesized by chemical synthesis, and pure product of 18 short peptides were prepared with a purity of more than 95%. Polypeptides were synthesized by Sichuan Pukang Pharmaceutical Co., Ltd. and confirmed by mass spectrometry and high performance liquid chromatography:

### 2) Comparison of Transdermal Performance

### 1. Preparation of Mouse Skin In Vitro

10 Kunming mice of 20 to 22 g with half male and half male were used in the experiment. After the mice were killed, the hairs on the abdomen were removed. Then the skin on the hair-removed area was peeled off, stripped of fat and grain, and rinsed repeatedly with distilled water. After being washed, the mouse skins were rinsed with normal saline, then treated with 10% glycerol, and stored at −20° C. for use (used up within 7 days).

### 2. Experimental Device

Single-chamber diffusion cell: The effective diffusion area of the diffusion cell is 2.0 cm², the volume of the receiving cell is 14 ml, the length of the custom-made stirrer is 1.4 cm, and the receiving liquid is 0.9% NaCl solution.

### 3. Preparation of Sample Solution

The stored mouse skin was taken out, thawed, washed with normal saline, and sandwiched between the receiving chamber and the supplying chamber, with the medicated side facing the supplying chamber and the skin side facing the receiving chamber. The temperature of the water bath system was adjusted to 37.5° C., and the stirring speed was adjusted to 100 rpm/min. 0.9% NaCl solution pre-warmed at 37° C. was added to the receiving chamber and the air bubbles were exhausted. In order to reduce the interference, the inner surface of the mouse skin was firstly contacted with the receiving solution without medication and all the receiving solution was replaced. Then the 18 collagen peptides synthesized were prepared into 5 mg/ml solutions using 0.9% NaCl respectively, which were injected into the supplying chamber and firmly adhere to the mouse skin respectively. After 24 h, part of the receiving solution was absorbed with a syringe as the sample solution to determine the transdermal amount of collagen peptide.

### 4. Detection of Target Peptide

The BCA kit method was used to determine the polypeptide content in the sample solution.

### 5. Results of Transdermal Absorption

TABLE 1
Comparison of the 24 h penetration amount
of different collagen peptides

| | | 24 h penetration |
|---|---|---|
| No. | Amino acid sequence | amount (ug) |
| 1 | gppgkngddgeagkpgrpger | 3041.21 |
| 2 | gergppgpqgarglpgtaglp | 3697.14 |
| 3 | gspgengapgqmgprglpger | 3213.66 |
| 4 | gergrpgapgpagargndgat | 3124.48 |
| 5 | gkrgargepgptglpgppger | 3023.05 |
| 6 | gerggpgsrgfpgadgvagpk | 3253.23 |
| 7 | gvmgfpgpkgaagepgkager | 3389.09 |
| 8 | gergvpgppgavgpagkdgea | 3113.64 |
| 9 | geqgvpgdlgapgpsgarger | 3341.78 |
| 10 | gergfpgergvqgppgpagpr | 2992.77 |
| 11 | gdlgapgpsgargergfpger | 2994.13 |
| 12 | gergvqgppgpagprgangap | 3103.23 |
| 13 | gapgapgsqgapglqgmpger | 4048.04 |
| 14 | gergaaglpgpkgdrgdagpk | 3745.15 |
| 15 | gpqgiagqrgvvglpgqrger | 3111.47 |
| 16 | gergfpglpgpsgepgkqgps | 3034.99 |
| 17 | glpgpsgepgkqgpsgasger | 3498.56 |
| 18 | gergppgpmgppglagppges | 3308.22 |

The results show that the polypeptide with the number of 13 and amino acid sequence of gapgapgsqgapglqgmpger (SEQ ID No.: 1) has the largest 24 h transdermal amount of 4048.04 ug.

### Example 2 Preparation of Various Type I Recombinant Collagens Using Escherichia coli Expression System

1) Preparation of Type I Recombinant Collagens with Different Repetition Times of SEQ ID No.: 1

The amino acid sequence of SEQ ID No.: 1 was repeated 3 times (P-1), 10 times (P-2), 20 times (P-3), 40 times (P-4) respectively, optimized by the codon preference of E. coli, and then translated into the corresponding gene sequences. The gene sequences were connected into the pET24a expression plasmids after the whole gene synthesis, and transferred into BL21 competent cells by thermal shock transformation to become expression strains (four kinds in total).

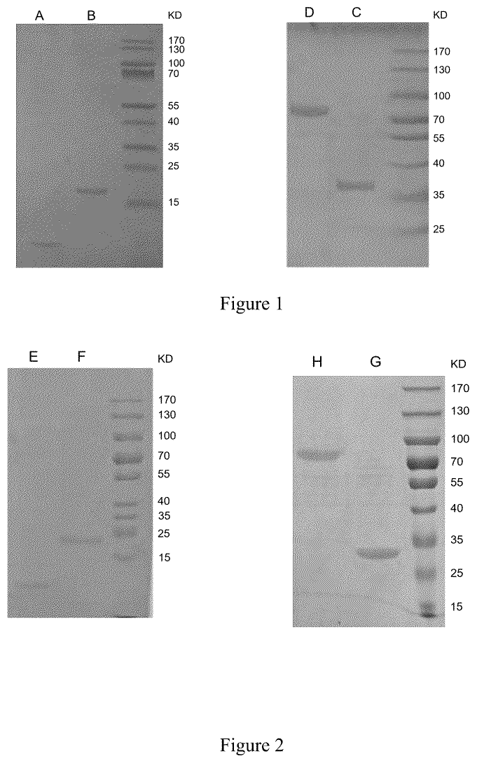

Single colonies of the above four kinds of expression strains were picked respectively, and transferred to LB liquid shake flasks to be cultured with shaking at 37° C. overnight to obtain the seed liquids. The seed liquids were transferred into 100 ml LB liquid mediums at 1% inoculum amount, cultured at 37° C. and 200 rpm, added with IPTG with a final concentration of 1.5 mM when the OD value was around 2 to 3, and cooled down to 28° C. for induction culture for 14 hours. The bacteria were collected by centrifugation, and prepared into 10% (wet weight of bacteria/PB volume) bacterial suspensions with PB buffer at pH 6.0, which were homogenized under high pressure of 1000 bar for 3 min. The supernatant were collected by centrifugation to obtain the crude protein expression solutions, which were purified by ion exchange chromatography. Proteins of 5.63 KD (P-1), 18.75 KD (P-2), 37.49 KD (P-3), and 74.96 KD (P-4) were collected respectively, which are type I recombinant collagens with different repetition times. The SDS-PAGE protein electrophoresis diagrams of various purified collagens are shown in FIG. 1, and lanes A, B, C, and D correspond to collagen P-1, P-2, P-3, and P-4, respectively.

2) Expression of Recombinant Type I Collagen Corresponding to Other Type I Collagen Amino Acid Sequences Containing GER Tripeptide with the Same Repetition Times

The amino acid sequence of No. 14 with better transdermal absorption performance in Table 1 was repeated 3 times (D-1), 10 times (D-2), 20 times (D-3), 40 times (D-4) respectively, optimized by the codon preference of E. coli, and then translated into the corresponding gene sequences. The gene sequences were connected into the pET24a expression plasmids after the whole gene synthesis, and transferred into BL21 competent cells by heat shock transformation to become the expression strains (four kinds in total).

Single colonies of the above four expression strains were picked respectively, and transferred to LB liquid shake flasks to be cultured with shaking at 37° C. overnight to obtain seed liquids. The seed liquids were transferred into 100 ml LB liquid medium with 1% inoculation amount, and cultured at 37° C. and 200 rpm, added with IPTG with a final concentration of 1.5 mM when the OD value was around 2 to 3, and cooled down to 28° C. for induction culture for 14 hours. The bacteria were collected by centrifugation, and prepared into 10% (wet weight of bacteria/PB volume) bacterial suspension with PB buffer at pH 6.0, which was homogenized under high pressure of 1000 bar for 3 min. The supernatant was collected by centrifugation to obtain the crude protein expression solution, which was purified by ion exchange chromatography. Proteins of 5.85 KD (D-1), 19.47 KD (D-2), 38.91 KD (D-3), and 77.81 KD (D-4) were collected respectively, which are recombinant collagens with different repetition times. The SDS-PAGE protein electrophoresis diagrams of various purified recombinant collagens are shown in FIG. 2, and lanes E, F, G, and H correspond to collagen D-1, D-2, D-3, and D-4, respectively.

### Example 3 Comparison of Transdermal Absorption Performance of Various Type I Recombinant Collagens

For the 8 kinds of recombinant collagens prepared in Example 2, permeated protein was measured by the method in comparison of transdermal performance in Example 1. The results are shown in Table 2.

TABLE 2
Comparison of the 24 h permeation amount of the recombinant
collages with different amino acid sequences

| No. | Recombinant protein | 24 h permeation amount/ug |
|---|---|---|
| 1 | P-1 | 2456.33 |
| 2 | P-2 | 1604.56 |
| 3 | P-3 | 1412.34 |
| 4 | P-4 | 1193.87 |
| 5 | D-1 | 2041.21 |
| 6 | D-2 | 1377.32 |
| 7 | D-3 | 1134.55 |
| 8 | D-4 | 813.24 |

As shown in Table 2, as the molecular weight increases, the transdermal absorption efficiency of the protein gradually decreases. However, the recombinant collagens formed by repeating SEQ ID No.: 1 screened in the present application show good transdermal absorption effect in recombinant collagen with similar molecular weight and similar repetition times.

1. A polypeptide derived from natural human type I collagen, having an amino acid sequence as shown in SEQ ID No.: 1 (GAPGAPGSQGAPGLQGMPGER).

2. A type I recombinant collagen, forming by multiple repetitions of a short amino acid sequence derived from natural human type I collagen as a repeating unit, wherein, the short amino acid sequence is shown in SEQ ID No.: 1 (GAPGAPGSQGAPGLQGMPGER), and times of the repetitions is three or more.

3. The type I recombinant collagen according to claim 2, wherein, the times of the repetitions is 5-150, preferably 10-100.

4. The type I recombinant collagen according to claim 2, further having a tag facilitating purification thereof, the tag being a His tag, a Flag tag or a c-Myc tag.

5. Use of the type I recombinant collagen according to claim 1 in the preparation of a collagen product.

6. The use according to claim 5, wherein the collagen product is selected from the group consisting of a collagen injection, a facial filler, a dressing, a cosmetic, a health food, a tissue engineering material and a collagen sponge.


SEQUENCE LISTING (1 sequence)

SEQ ID NO: 1 - protein, 21 aa, Homo sapiens
GAPGAPGSQGAPGLQGMPGER